(19) United States
(12) Patent Application Publication
(10) Pub. No.: US 2008/0199943 A1
(43) Pub. Date: Aug. 21, 2008

(54) Material Solubiliser Reactor For Hydrolysis and/or Wet Fermentation and Waste Treatment Plant With Such a Solubiliser and Reactor

(76) Inventors: Christian Widmer, Binningen (CH); Rudolf Hartman, Gelterkinden (CH); Hans Wuthrich, Kienberg (CH)

Correspondence Address: BOYLE FREDRICKSON S.C., 840 North Plankinton Avenue, MILWAUKEE, WI, 53203, US

(21) Appl. No.: 11/628,353

(22) PCT Filed: Jun. 3, 2005
(86) PCT No.: PCT/EP2005/005993

§ 371 Date: Dec. 21, 2007

(30) Foreign Application Priority Data
Jun. 3, 2004 (DE) 10 2004 027 260.3
Oct. 15, 2004 (DE) 10 2004 050 503.9

(51) Int. Cl. C12P 1/00; C12M 1/00

(52) U.S. Cl. 435/262; 435/290.2

(57) ABSTRACT

The invention relates to a method for the treatment of waste with organic components, whereby in standardized method steps, various material solubilisers, for dissolving the organic material in a solvent and various reactors for carrying out a hydrolysis and/or a wet fermentation are used depending on the particle size and suitable solubilisers and reactors. A suitable waste treatment plant is also disclosed.

### BACKGROUND OF THE INVENTION

1. Field of the Invention

The invention relates to a method for the treatment of waste with organic components, a material solubilizer for dissolving organic components of waste in a solvent, a reactor for carrying out a hydrolysis and/or a wet fermentation, as well as a waste treatment plant containing such material solubilizers and/or reactors.

2. Description of Related Art

Upon introduction of the separate collection of organic household refuse in Europe, the mechanically biological recovery (German abbreviation: MBA) of urban refuse has become increasingly important. The decomposition of the biogenic mass takes place on a microbial basis, wherein a difference can be made between aerobic and anaerobic microorganisms. The aerobic reaction ultimately results in the final products carbon dioxide and water and is referred to as rotting. The anaerobic reaction is typical of fermentation; the final products formed are, inter alia, methane, ammonia and hydrogen sulfide.

Known methods provide various method steps for waste treatment depending on the nature of the waste mixtures. The individual provision of individual method plants is very expensive, however.

In DE 196 48 731 A1 an aerobic method is described in which the organic components of a waste fraction are washed out in a percolator and the residue is burnt or deposited, for instance, after drying.

The percolation can be carried out, for example, in a box percolator according to WO 97/27158 A1. Also tests using a boiling percolator according to DE 101 42 906 A1 in which the percolation is carried out in the boiling range of the process water turned out to be promising.

The organically highly loaded exit water extracted from the percolator is supplied to a biogas plant for anaerobic decomposition, wherein the organic part is reacted by means of methane bacteria and can be fed to biogas combustion for energy generation. The afore-described aerobic treatment of the waste materials in a percolator has turned out to be extremely competitive with the anaerobic methods and has become increasingly important.

In EP 0 192 900 B1 the Valorga method, as it is called, is described—in which the fermentation is carried out in a fermenter which is charged from the bottom. The waste to be recovered is guided in plug shape to an outlet arranged below the radially outer inlet opening. The waste is conveyed by blowing in compressed biogas via gas nozzles disposed in several sectors of the fermenter, wherein each sector can be individually controlled to maintain the plug flow of the waste between the inlet opening and the outlet opening.

In EP 0 476 217 A1 a heatable fermenter is disclosed in which starting material and sludge material are supplied to the fermenter as bacteria inoculum and the sludge material formed is transported to a sludge material outlet via an agitator. Such an addition of inoculum may also be provided in the Valorga method according to EP 0 192 900 B1 described in the beginning.

EP 0 794 247 A1 discloses a fermenter in which the fermented product is introduced to a rotating drum in which a spiral is arranged. The fermented product is guided in plug shape from the inlet to the sludge material outlet via said spiral. This supply can take place by forward and backward rotation of the drum, wherein the forward rotation, i.e. the transportation of the fermented product in the direction of the fermented product outlet takes longer than in the opposite direction so that a predetermined holding time of the fermented product is reached.

In the above-described known methods dry waste is treated which has comparatively high dry matter content (TS) of more than 25%.

When treating fluid humid waste, for instance, according to DE 197 04 065 A1, so-called solubilizers (pulpers) in which the waste is diluted with a solvent and is torn apart and crushed by means of a mixer so that a suspension is formed and organic material dissolves in the solvent. In the known solution the mixing is carried out by means of an agitator the blades of which are designed such that a vertical flow is formed in sections in the solubilizer. It is a drawback of this solution that, on the one hand, a considerable expenditure on apparatuses for forming the complex geometry of the agitating blades is required and, on the other hand, said blades are subjected to considerable wear due to the floating material and impurities contained in the suspension.

In DE 196 24 268 A1 a fermenting method for waste in fluid form is disclosed. A multi-chamber reactor is used for this purpose, wherein the fermented product can be transported from an inlet opening through the chambers to an outlet opening via an agitator. A common gas chamber from which the biogas formed during the fermenting process is extracted is allocated to the multi-chamber reactor. The metabolism can be individually controlled in the individual chambers by a different conduct of the process, for instance via heat exchangers, addition of inoculum etc.

Since the waste to be treated also contains a quite considerable part of high-gravity solids and impurities, especially the solutions using mechanical conveying means (EP 0 794 247 A1, EP 0 476 217 A1, DE 197 04 065 A1, DE 196 24 268 A1) are subjected to relatively high wear, because the conveying means employed and other internal parts can be damaged by the sediments including the impurity/high-gravity solids.

### SUMMARY OF THE INVENTION

Compared to the above, the object underlying the invention is to provide a uniform method of treating waste with organic components. Furthermore it is the object of the present invention to provide solubilizers and reactors for use in such method as well as a respective waste treatment plant.

This object is achieved by a method comprising the features according to claim 1, a solubilizer comprising the features of claim 5 and 19, respectively, a reactor comprising the features of claim 28 and 36, respectively, as well as by a waste treatment plant comprising the features of claim 41.

A method preferred according to the invention includes a mechanical treatment of the waste, dissolution of organic material in a solubilizer, hydrolysis of the biological loaded suspension withdrawn from the solubilizer in a reactor and fermentation, wherein the process water obtained during hydrolysis or in the fermenter is circulated as circulating water. In accordance with the invention, depending on the particle size of the mechanically prepared waste mixture the solubilizer and/or reactor to be used in the plant is selected. This has the advantage that the method is identical for different waste mixtures and only the plant parts of material solubilizer and reactor have to be chosen in dependence on the particle size of the waste. A preferred “limit particle size” is approx. 80 mm.

Advantageously, in addition to the hydrolysis a wet fermentation or wet oxidation is provided which is carried out in a reactor corresponding to the hydrolysis reactor.

For introducing a biological suspension substantially freed from solid matter into the fermenter suitable separating steps for separating impurities, high-gravity solids, fibrous material etc. can be provided.

In accordance with the invention, the organic material having a maximum particle size of approx. 80 mm is dissolved in a material solubilizer including, instead of a known mechanical agitator, a so-to-speak pneumatic agitator in which by injecting gas, preferably air, the suspension is mixed in the solubilizer and the organic material passes as solution into the solvent by which a suspension flow is generated in the solubilizer.

This pneumatic solution has practically no wear and can be realized with a considerably lower expenditure on apparatuses than it is the case with the conventional solutions. It turned out that the organic material can be dissolved in a considerably shorter time than in the constructional designs including a mechanical agitator.

The thorough mixing can be further improved when the gas injecting nozzles are part of a gas flow pump by which the suspension can be recirculated periodically or continuously inside the solubilizer tank. The gas may also be injected in the bottom of the solubilizer tank so that also the impurities/high-gravity solids accumulating there are mixed with the gas.

Said gas flow pump preferably includes in inner pipe at the lower end portion of which a nozzle plate having gas injecting nozzles through or around which the suspension can flow is arranged and at the upper end portion of which an outlet opening for the suspension conveyed in the inner pipe is formed.

In the case of a particularly efficiently operating embodiment, at a distance from the outlet opening a bounce plate is arranged against which the material mixture conveyed by the gas flow pump rebounds at high velocity and is decomposed. The organic material is converted into the water phase. Inert material particles and sand settle at the bottom and can be removed. Fibrous material and solid matter components contained in the suspension rub against one another during said conveyance toward the bounce plate and are additionally freed from persistent organic components.

In a preferred embodiment the bounce plate delimits in sections a gas discharge chamber by which the gas guided in the circulation is discharged.

In the case of large tank volumes it may be advantageous to arrange plural gas flow pumps in the material solubilizer tank.

According to the invention, it is especially preferred when the inner pipe is double-walled, wherein the gas injecting nozzles then are arranged either in the inner cylinder chamber or in the annular chamber and the respective other chamber serves for accommodating a heating medium so that the inner pipe simultaneously acts as heat exchanger by which the suspension is kept at a processing temperature.

The thorough mixing can be further improved when deflector plates are arranged at the outer circumference of the inner pipe for guiding the flow. Since said deflector plates are fixedly disposed in the material solubilizer tank, the wear thereof is minimal.

In particular applications it may be advantageous to operate plural material solubilizers in series.

A material solubilizer according to the invention for dissolving organic components of waste having a minimum particle size of approx. 80 mm in a solvent provides, according to the invention, at least one mechanical agitator whose respective adjacent agitating members have opposed conveying directions. This has the advantage that the mixture provided in the material solubilizer is conveyed between the agitating members toward each other and away from each other so that an improved abrasion and thus an improved dissolution of the organic material can be obtained.

Preferably the agitating members are rotor blades arranged on a rotor the blade pitch angles of which are offset with respect to each other by approx. 180°. The number of rotor blades is freely selectable, but an even number, for instance 6 rotor blades, is preferred.

The rotor blades can be evenly distributed on the rotor from an inlet lock for the waste to an outlet opening for separated impurities/high-gravity solids.

It is equally possible that the material solubilizer has plural parallel rotors, the rotor blades of the individual rotors forming a respective overlapping area.

In a particular embodiment, a gas injection for swirling the impurities/high-gravity solids can be arranged in the area of the extracting opening. It is possible in this context that the injected gas is guided in the circuit so that the amount of gas required is reduced.

The material solubilizer may be rectangular in longitudinal section, wherein its length L1 corresponds at least the fourfold height h1.

According to the invention, during hydrolysis and/or wet fermentation of the suspension from the waste with a maximum particle size of approx. 80 mm a reactor is used having a mechanical mixer for mixing the material mixture and a draft tube enclosing the mixer. The mixer is controlled such that the material mixture can be sucked from a reactor head side to the reactor bottom side through the deflecting pipe, wherein an ascending loop-shaped flow is formed outside the draft tube.

For optimizing the hydrolysis and/or wet fermentation the draft tube has an axial extension for varying its length and/or height. Furthermore plural draft tubes, for instance 3 draft tubes, having an appropriately reduced diameter can be arranged in a reactor.

The oxygen required for hydrolysis and/or wet fermentation can be supplied through oxygen blowing in the vicinity of the bottom and/or in the area of the mixer.

For controlling the amount of oxygen to be blown in an O₂ probe detecting the O2 content can be provided so that, in response to these signals, the axial extension, the axial position of the draft tube and/or a material mixture level are adjustable such that preferably an optimum, i.e. almost 100% oxygen utilization takes place.

Exemplary geometrical relations are e.g.:

The height of the draft tube H1 corresponds to 8 to 10 times the diameter d1 of the draft tube,

the active diameter d2, i.e. the inner diameter of the reactor, corresponds to 4 to 6 times the diameter of the draft tube d2,

the bottom distance H2 from the reactor bottom to the draft tube corresponds to 1 to 2 times the diameter of the draft tube d1,

the distance between the material mixture level and the draft tube corresponds to 2 to 3 times the diameter of the draft tube d1,

the variable height adjustment H4 between the material mixture level and the draft tube is 0.5 to 2 times the diameter of the draft tube d1,

the upstream velocity v1 of the circulating flow ranges between 0.1 m/s and 0.8 m/s,

the diameter of the draft tube d1 is between 0.5 m and 1.5 m depending on the material mixture composition and the dry matter content.

Overheating of the material mixture can be efficiently prevented by a cooling medium flowing past the draft tube.

Basically plural hydrolyses or wet fermentations can be arranged in series.

A reactor according to the invention for treating a supplied suspension loaded with organic material obtained from a waste mixture having a minimum particle size of approx. 80 mm comprises a blowing means for gas, preferably oxygen, as mixing means for mixing the material mixture.

Gas is preferably injected via a plurality of gas injecting nozzles near the bottom of the reactor and is controllable via a gas measuring probe.

The gas is preferably adapted to be circulated via a pump in the circuit.

In order to increase the thorough mixing, gases formed in the reactor can likewise be injected to the material mixture near the bottom of the motor via a blower.

A waste treatment plant designed to comprise the material solubilizer preferably includes a solid matter treatment for separating and washing the impurities/high-gravity solids extracted from the material solubilizer.

In accordance with the invention, the waste treatment plant can also comprise a separating step for depositing fibrous material or the like from the decomposed suspension removed from the material solubilizer. Said separating step preferably includes a washing plant and a dehydrating press by which the deposited fibrous/floating substances can be cleaned and supplied to further use.

In addition to the fibrous material separators, the waste treatment plant may be designed to include sand washing for washing fine sand which is still contained in the remaining suspension (solvent) after separating the fibrous material.

The solvent containing the organic material is preferably supplied to a fermenter in which said organic material is converted into biogas and/or supplied to wet fermentation or wet oxidation as mixing water.

The solvent freed from the organic material is then returned to the material solubilizer, wherein excessive water can be supplied to a waste water purification plant.

The solids content supplied to the material solubilizer is preferably minimized by a solids treatment connected upstream.

It has turned out that the holding time through the treatment plant according to the invention can be reduced from usual approximately 61 days to approx. 29 days, when the decomposed suspension of the material solubilizer undergoes a hydrolysis at least as partial flow and is subsequently freed from fibers and solids, the solids passing the wet fermentation or wet oxidation at least as partial flow for obtaining an oxidized material mixture.

During hydrolysis the suspension of the material solubilizer is aerobically acidified and the not yet decomposed organic material is likewise decomposed so that additional material can be supplied to the fermenter.

It is equally possible to subject at least a partial flow of the solids separated in a separating plant connected downstream of the hydrolysis to drying and compacting for the preparation of molded parts for gasification and combustion plants. Preferably the compacting is carried out at lower pressure and by adding a binder acting as adhesive until glowing away in the gasification and combustion plant. The binder can be self-produced during waste treatment, i.e. from separated plastic material, or can be supplied.

For the gasifying operation the molded parts must remain “gasification-stable” in the glowing state, i.e. the shape is retained up to the incineration.

In an embodiment of a treatment plant the suspension treated during hydrolysis is directly supplied to the fermenter. Since the unloaded waste water then resulting during fermentation still may have a high solids content, it should not be added to the diluting or circulating water. An admixture can be obtained, however, by substantially separating the solids in a separating plant from the waste water so that the waste water is free from solids. The dehydrated solids then can be subjected to wet fermentation, wherein a partial flow of the waste water free from solids can be mixed with the solids again to form a suspension for an optimum adjustment of the solids content.

In a different embodiment of a treatment plant the solids get from the hydrolysis to the wet fermentation or wet oxidation, respectively. By exposure to oxygen the organic material which cannot be anaerobically decomposed is respired and the nitrogen is expelled as ammonia.

The material mixture oxidized after the wet oxidation can be supplied to a separating plant comprising a solids decomposer, a solids screening and washing plant as well as a dehydrating press. It is possible to use the waste water resulting from the solids decomposer as solvent for the material solubilizer and/or to supply it to the waste water purification plant. The raw compost formed in the dehydrating press can be directly disposed of.

Preferably mixed water resulting from the mixing of the circulating water with the waste water of the fermenter is supplied to the material mixture during wet fermentation.

The oxidized material mixture resulting from wet fermentation can pass through a separating plant for producing raw compost and waste water. The waste water can be mixed with the solvent and/or fed into the waste water purification plant. The raw compost can be subjected to subsequent rotting for drying and/or can be directly disposed of.

The waste gases formed during hydrolysis and wet fermentation can be supplied to a pneumatic washer to free them from ammonia.

The treatment plant comprises especially for mechanically treated waste mixtures having a maximum particle size of approx. 80 mm the material solubilizer according to the invention including pneumatic agitator and for hydrolysis and/or wet fermentation the reactor according to the invention including a mechanical agitator.

For mechanically treated waste mixtures having a minimum particle size of approx. 80 mm preferably the material solubilizer according to the invention including mechanical agitator is used and for hydrolysis and/or for wet oxidation the reactor according to the invention including pneumatic agitator is used. The latter can also be used for the smaller particle sizes. The “limit particle size” may vary depending on the waste to be treated, said 80 mm are to be considered as example.

Advantageously, at least in the reactor for wet oxidation a hygienization of the material mixture can be carried out in the reactor in an appropriate operating mode.

For freeing the waste gases formed during hydrolysis and wet oxidation from ammonia a pneumatic washer for washing out the ammonia can be provided.

Other advantageous further developments of the invention are the subject matter of further subclaims.

### BRIEF DESCRIPTION OF THE DRAWINGS

Hereinafter preferred embodiments of the invention are explained in detail by way of schematic drawings, in which

FIG. 1 shows a schematic diagram of a material solubilizer according to the invention for waste mixtures having an approximate particle size of less than 80 mm;

FIG. 2 shows a schematized cross-section of the material solubilizer from FIG. 1;

FIG. 3, FIG. 4 show cross-sections of alternative embodiments of a material solubilizer;

FIGS. 5 to 7 are schematic diagrams of different operating states of the material solubilizer from FIG. 1;

FIG. 8 is a variant of the material solubilizer according to FIG. 1;

FIG. 9 shows a waste treatment plant comprising a material solubilizer according to FIG. 1,

FIG. 9a shows an alternative operating case from FIG. 9 in a simplified and enlarged representation (cf. also FIG. 19),

FIG. 9b shows a further alternative operating case from FIG. 9 in a simplified and enlarged representation (cf. also FIG. 19),

FIG. 10 is a detailed representation of a hydrolysis and a wet fermentation from FIG. 9;

FIG. 11 shows two material solubilizers from FIG. 1 connected in series;

FIG. 12 is a longitudinal section across an alternative material solubilizer according to the invention for waste mixtures having an approximate particle size of more than 80 mm;

FIG. 13a to 13d show exemplary cross-sections across the material solubilizer according to FIG. 12;

FIG. 14 is a series connection of the solubilizer from FIG. 12;

FIG. 15 shows a longitudinal section across a preferred embodiment of a reactor for hydrolysis or wet fermentation for waste mixtures having an approximate particle size of less than 80 mm;

FIG. 16 shows a cross-section across a further preferred embodiment of a reactor for hydrolysis or wet fermentation;

FIG. 17 shows a series connection of plural reactors during hydrolysis, and

FIG. 18 shows a series connection of plural reactors during wet fermentation;

FIG. 19 is a simplified process diagram of the waste treatment plant according to the invention;

FIG. 20 shows a hydrolysis reactor for waste mixtures having an approximate particle size of more than 80 mm;

FIG. 21 shows an alternative wet fermentation reactor for waste mixtures having an approximate particle size of more than 80 mm;

FIG. 22 shows a detailed material separating plant from FIG. 19;

FIG. 23 shows a detailed separating plant from FIG. 19 and

FIG. 24 shows a detailed process diagram of compacting from FIG. 19.

### DETAILED DESCRIPTION OF THE PREFERRED EMBODIMENT

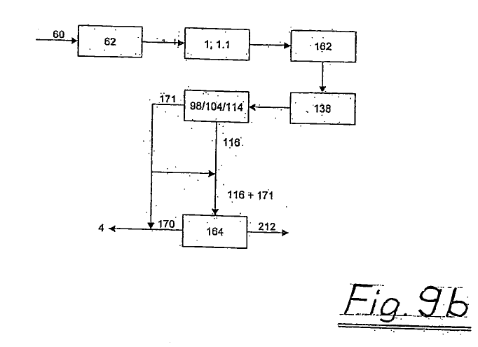

In FIG. 1 the basic structure of a material solubilizer 1 is shown in which organic material of a supplied input material 2, preferably waste in a solvent, for instance diluting water 4, is dissolved so that in the material solubilizer 1a mixture 8 is provided having a dry matter content of about 5 to 10%. The waste mixture supplied to the solubilizer 1 preferably has a maximum particle size of approx. 80 mm. The waste 2 and the diluting water 4 are supplied via inlet locks 10 to a material solubilizing tank 6. A bottom 12 of the material solubilizing tank is tapered and opens in a discharge opening 14 having an outlet lock 16 through which impurities/high-gravity solids 18 settling at the tapered bottom 12 can be extracted. In the area of the tapered bottom 12a further outlet lock 16 is formed through which the suspension 20 decomposed in the solubilizer 1 and loaded with organic material is extracted and, according to FIG. 9, treated and then supplied to the circuit again as diluting water 4 via the inlet lock 10.

Inside the material solubilizing tank 6a gas flow pump 24 is arranged by which the mixture 8 is mixed inside the material solubilizing tank—as will be described in detail hereinafter. In the embodiment shown in FIG. 1 the gas flow pump 24 includes an inner pipe 26 arranged coaxially with respect to the material solubilizing tank 6 and having a nozzle plate 27 including a plurality of gas injecting nozzles 28 at its lower inlet opening in FIG. 1 through which a gas, preferably air, can be injected. A suspension 8 can flow around the nozzle plate 27. The gas injecting nozzles 28 are connected via a compressed-air line 30 and a control valve 32 controllable by the plant control to a medium-pressure reservoir or air vessel 34 charged to a pressure of 3 to 8 bar, for example, by an pneumatic compressor 36. The latter sucks transport air 40 from a gas discharge chamber 42 at the head 22 of the material solubilizing tank 6 via a suction line 38—i.e. said transport air 40 is likewise guided in the circuit and by appropriate control of the control valve 32 is pressed from the air vessel 34 via the compressed-air line 30 and the gas injecting nozzles 28 into the inner pipe 26.

Via a change-over means and/or dosing means 66 downstream of the pneumatic compressor 36 the air vessel 34 can be bypassed by the control valve, i.e. the pulsation. In this context, a bypass line 154 which downstream of the control valve 36 opens in the compressed-air line 30 is controlled to open. In this case the mixture 8 can be circulated by the blower pressure corresponding to 1.5 times the pressure height.

Furthermore a change-over and/or dosing means 66 from which an injecting line 156 extends into the discharge opening 14 of the material solubilizer 1 can be provided in the compressed-air line 30. Thus also the impurities and high-gravity solids can be moved and mixed by compressed air so that the adhering organic material separates and goes into the mixture 8.

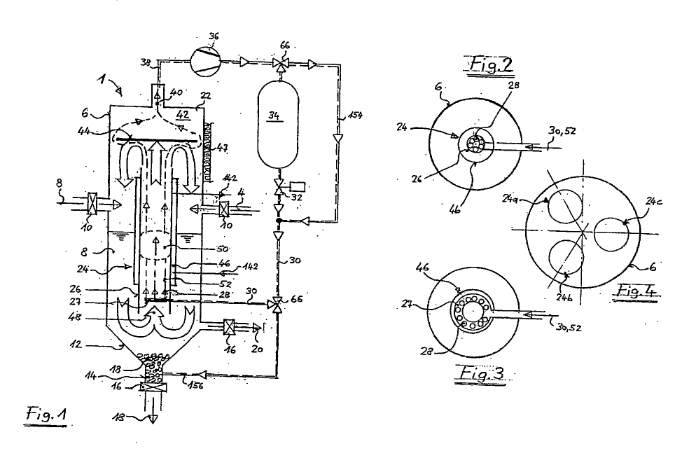

FIG. 2 schematically shows the cross-section of the solubilizing tank 6 including the concentrically arranged gas flow pump 24 in which the inner pipe 26 is provided with a double shell 46 through which the so-called heating medium flows. The gas injecting nozzles 28 are disposed in the inner cylinder chamber enclosed by the inner pipe 26.

In an alternative variant according to FIG. 3 the gas injecting nozzles 28 can also be disposed in the annular chamber enclosed by the double shell 46 so that the heating medium flows through the central cylindrical chamber.

In the case of very large tank volumes it may be advantageous to arrange plural, for instance three gas flow pumps 24a, 24b, 24c in the material solubilizing tank 6.

At a distance above an outlet opening of the inner pipe 26a bounce plate 44 is disposed which delimits the gas discharge chamber 42 downwards in sections and the transport air 40 can laterally flow around it.

For heating the suspension 8 to the process temperature the inner pipe 26 is provided with a double shell 46, wherein a heating medium is guided in the resulting annular chamber so that the inner pipe 26 acts as heat exchanger. The shell of the material solubilizing tank 6 can be provided with insulation.

For solubilizing the material, the input material 2 introduced to the material solubilizing tank 6 is initially adjusted to a dry matter content (German abbreviation: TS) of approx. 5 to 10% by supplying the diluting water 4 guided in the circuit. Subsequently compressed air is injected through the gas injecting nozzles 26 by controlling the control valve 32. In the shown embodiment a pulsating operation is preferred, wherein the pulse distance is about 5 to 10 seconds, for instance. The processing temperature is adjusted to a temperature between 50 and 70°via the heating medium flowing in the double shell 46. Due to this compressed-air pulsation in the interior of the gas flow pump 24, compressed-air bubbles 50 which, similarly to a piston of a piston pump, suck mixture/suspension 8 from the bottom 12 so that inside the inner pipe 26 an upwardly directed suspension flow 48 is formed. Said sucked suspension then impinges on the bounce plate 44 at high velocity which may be within the range of from 10 to 20 m/s, wherein a mechanical decomposition takes place by the rebounding and frictional energy and the organic material goes into solution in the diluting water 4.

The compressed air 52 flowing through the inner pipe 26 flows around the bounce plate 54 and is then most largely relieved in the area of the gas discharge chamber 42 and is sucked by the compressor 36 as transport air 40 and is supplied to the air vessel 34 again—the compressed air circuit is closed.

Inert material particles, sand, impurities/high-gravity solids etc. contained in the waste are dissolved and settle toward the tapered bottom 12. Moreover, fibrous material is released and goes into suspension, wherein films and other solid material are cleaned from adhering organic material by the introduced shear forces. The forming impurities/high-gravity solids are extracted through the outlet lock 16 and the outlet opening 14 at the bottom 12 of the material solubilizing tank 6. It turned out that by the material solubilizer the organic material can be brought into solution by far more quickly and with lower expenditure on apparatuses than this is the case by conventional solubilizers in which mechanical agitators and the like are used.

The function of the gas flow pump 24 is illustrated in detail once again by way of the FIGS. 5 to 7.

In FIG. 5 the material solubilizing tank 6 is shown in a filled idle state, wherein in the same the input material 2 is adjusted to said dry matter content of 5 to 10% by addition of diluting water 4. A level 54 of the material mixture is adjusted such that is lies below the upper outlet opening of the inner pipe 26 of the gas flow pump 24. By the pneumatic circulation explained by way of FIG. 1 suspension is sucked by the upwardly directed suspension flow 48 and is thrown against the bounce plate 44 and then flows downwards again in the annular chamber delimited by the inner pipe 24 and by the shell of the solubilizing tank 6. The part of the upwardly conveyed suspension is so large that the level 54 inside the solubilizing tank 6 settles by the measure Ah according to FIG. 6. When the air injection is finished, i.e. after the end of each compressed-air pulse, the suspension column sinks downwards inside the inner pipe 26 (see FIG. 7) and the level 54 in the annular chamber 56 rises again until the initial state according to FIG. 5 is adjusted—the next injecting cycle can start. By the afore-described flows inside the material solubilizing tank 6 and by the impinging of the suspension on the bounce plate 44 the suspension is extremely intensely mixed so that the organic components of the input material 2 are brought into solution very quickly and with a high efficiency and, moreover, the fibrous material is suspended and the impurities/high-gravity solids are settled. Since practically no moving components are required for this intense mixing inside the material solubilizing tank 6, the wear of the material solubilizer 1 according to the invention is minimal vis-à-vis conventional solutions.

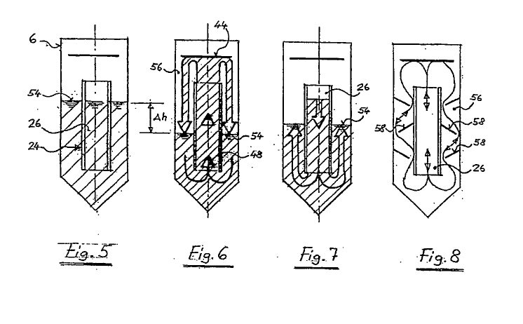

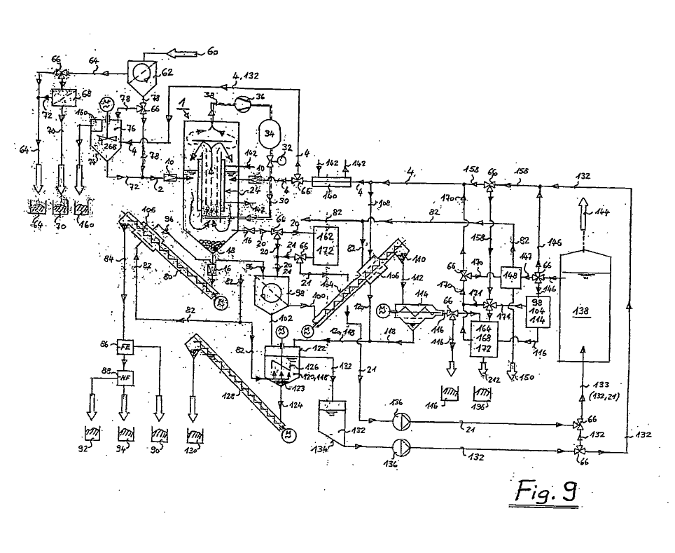

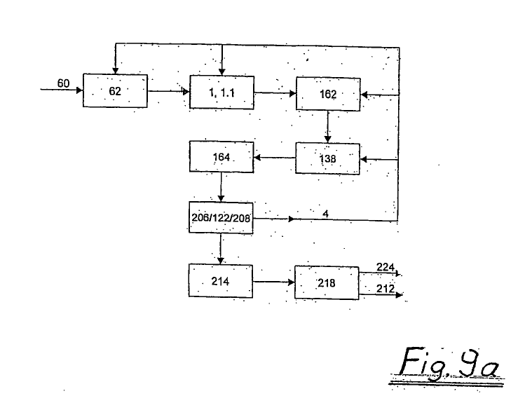

The mixing can be further improved when, according to FIG. 8, internal parts, for instance downwardly inclined deflector plates 58 around which the downwardly directed suspension flow (FIG. 6) has to flow, are provided in the annular chamber 56 so that further shear forces are introduced to the suspension. As said deflector plates 58 are arranged in a stationary manner, the wear thereof is equally minimal. In the embodiment shown the deflector plates 58 are alternately disposed at the inner circumferential shell of the solubilizing tank 6 and at the outer shell of the inner pipe 26 so that the shown wave-shaped flow is resulting in the annular chamber 56. Of course, instead of the deflector plates 58 also other internal parts or fillers can be used.

In FIG. 9 a waste treatment plant is shown in which the afore-described material solubilizer 1 according to FIG. 1 is used.

In this waste treatment plant several steps for separating solids are arranged ahead of the material solubilizer 1. The waste 60 to be treated is first of all—after crushing where appropriate—supplied to a screening plant 62 which is a rotating screen in the shown embodiment. The screen overflow 64 having a particle size of from 80 to 200 mm is then eliminated either directly by a material distributing guide or a change-over and/or dosing means 66 or is separated by an additional step. In the shown embodiment, a partial flow or the entire solids flow can be guided via the change-over and/or dosing means 66 to a pneumatic classification plant 68 in which the screen overflow 64 is separated into high-gravity solids/impurities 70 and soiled light solids 72 which are eliminated.

The underflow 78 rich in organic material can be supplied by a change-over and/or dosing means 66 to a mixing plant 74 in which it is diluted with a partial flow of the NOₓ reduced diluting water 4 and treated by means of a mixer 268 to form a suspension 76 having a solids content of 5 to 15%.

The suspension 76 is supplied to the inlet lock 10 of the material solubilizer 1. Impurities 160, such as ribbons, ropes and cables, are separated from the suspension 76 and discharged via a mechanical device of the mixing plant 74.

The impurities/high-gravity solids 18 formed in the material solubilizer 1 are extracted from the solubilizer 1 through the outlet lock 16 and are supplied to a washing means 80 in which they are cleaned from adhering organic material in a purification zone 106 by means of supplied process water 82. The cleaned high-gravity solids/impurities 84 are then fed to a ferrous metal separator 86 as well as a non-ferrous metal separator 88 so that the material flow 84 is appropriately divided into a ferrous part 90, a non-ferrous part 92 and other material 94.

The decomposed suspension extracted from the material solubilizer 1 through the outlet lock 16 is supplied along with the soiled process water 96 from the washing means 80 to a fibrous material separator 98 which, in turn, is in the form of a rotating screen. In said fibrous material separator 98 fibers and floating substances 100 are separated from water containing organic material 102. The fibrous/floating substances 100 are cleaned in a solids screening and washing plant 104 by the addition of process water 82 which is supplied to a purification zone 106 of the washing plant. Said purifying operation can be additionally assisted by supplying to the purification zone 106 circulating water 108 which is branched off the treatment circuit for the diluting water 4.

In the described embodiments each of the two washing means 80, 104 is designed to include obliquely inclined spiral conveyors via which the respective material flow to be cleaned is conveyed to one of the purification zones 106 and finally extracted through a solids outlet 110. In the purification zone 106 organic material is dissolved from the solids. In the event that a very intense purification is required, said purification is substantially carried out by process water 82, in the case of lower requirements to said purification the share of circulating water 108 can be increased.

The solids and fibers 112 cleaned and extracted through the solids outlet 110 of the washing plant 104 are then dehydrated in a dehydrating press 114 and the dehydrated solids 116 are supplied to thermal utilization or a subsequent rotting for later disposal.

The water 118 resulting from the dehydrating press 114 and containing dissolved organic material is subsequently mixed with the rinsing water 120 flowing off the purification zone 106 and loaded with organic material. Said material flow contains a share of fine sand which is separated in a sand washer 122. Also the water 102 containing organic material from the fibrous material separator 98 is supplied to the material flow. In the sand washer the fine sand component 124 is separated by the action of an agitator 126, is discharged via a sand discharge 123 and cleaned from adhering organic material by the addition of process water 82. The pre-cleaned fine sand 124 is then supplied to a fine sand washing means 128 the basic structure of which corresponds to the washing means 80, 104 so that further remarks can be dispensed with. The cleaned fine sand 130 can then be fed to a material utilization in civil engineering and road construction.

The organically highly loaded circulating water 132 provided after sand washing is then intermediately stored in an intermediate reservoir 134 and is either supplied to a fermenter 138 by means of a pump 136 or is directly fed as circulating water 132 to a heat exchanger 140 in which it is heated to the process temperature by a heating medium 142 and after that is introduced to the material solubilizer 1 as diluting water 4 through the inlet lock 10. The heating medium 142 can also be used for heating the double shell of the gas flow pump 24.

Depending on the process control, the organic material of the water supplied to the fermenter 138 is converted to biogas (methane gas) 144 by methanation.

The waste water 146 provided after the fermentation step and freed from organic material is then mixed with the possibly provided circulating water 132 and brought to processing temperature in the heat exchanger 140. Excessive water 147 not required in the circuit is supplied to a waste water treatment plant 148 and the cleaned waste water 150 is discharged and guided into the sewerage system. A partial flow of the cleaned waste water 150 is guided as process water 82 to the washing means 80, 104, 128 as well as to the sand washer 122 so that also the process water circuit is closed.

Organic material contained in the decomposed suspension 20 can be separated from the waste even more quickly, when the decomposed suspension 20 of the material solubilizer 1 is first supplied to an aerobic hydrolysis or acidifying step 162 via a change-over and/or dosing means 66 and after a treatment period of 1 to 4 days the suspension 20 is free from solids in the fibrous material separator 98 and the sand washer 122. Subsequently, the suspension 21 treated in this way is stored in the intermediate reservoir 13 as organically highly loaded circulating water 132 and supplied to the fermenter 138.

The separated solids and fibers 100 of the fibrous material separator 98 which subsequently pass through the solid screening and washing plant 104 and the dehydrating press 114 is supplied to a wet fermentation 164 via a change-over and/or dosing means 66 as dehydrated solids 116 having a dry matter content of 35% to 60% TS and are there diluted with the mixing water 158 to a dry matter content of 5 to 15% via a change-over and/or dosing means 66.

After a holding time of 3 to 10 days in the wet fermentation 164, the oxidized and NOₓ reduced material mixture 23 is extracted and freed from solids in a separating plant 168. The resulting waste water 170 which is almost free of solids is then supplied as diluting water 4 to the material solubilizer 1 and/or via a change-over and/or dosing means 66 to the waste water treatment plant 148. The resulting raw compost 212 is disposed of.

The waste gases formed during hydrolysis 162 and during wet fermentation 164 are jointly freed from ammonia in an acid pneumatic washer 172.

The organic components of the waste can be separated by means of the above-described waste treatment plant with a very low expenditure on apparatuses and the remaining material flow can be separated into recyclable or disposable partial material flows.

In accordance with FIG. 9, it is likewise possible in an operating case to bypass the separating means 98, 104, 114, 122, 128 and to supply the suspension 21 prepared in the hydrolysis 162 directly to the fermenter 138, wherein a suspension mixture 133 is produced of the organically highly loaded waste water 132 and the prepared suspension 21 via a change-over and/or dosing means 66. The solids-bearing waste water 146 of the fermenter 138 is supplied to the wet fermentation or wet composting 164 as fermenting material via a change-over and/or dosing means 66.

According to FIG. 9a the oxidized material mixture 23 is then subjected after wet fermentation 162 to a material separation including a filter means 206, a sand washer 122 and a dehydration press 208 for separating the solids. The solids-free waste water 170 obtained during material separation is used as solvent or circulating water 4. The solids 212 separated during material separation can be subjected to subsequent rotting 214, wherein the dry product 216 resulting from subsequent rotting 214 passes through screening 218 in which the residual materials 224 and the compost 212 are separated. The residual materials are supplied, e.g., to material-sensitive recycling.

When the fermenter 138 is primarily charged with the solids-bearing suspension 21 after hydrolysis 162, the waste water 146 loaded with solids can only be introduced to the circuit of the diluting water 4, if, as indicated in FIG. 9 and shown enlarged in FIG. 9b, the solids and fibers were separated before in a separating plant including solids separation 98, solids screening and washing plant 104 and subsequent dehydrating press 114. The control of the waste water 146 is carried out by a change-over and/or dosing means 66. The solids fermented in the fermenter 138 and separated in the separating plant 98, 104, 114 are supplied to the wet fermentation 164, wherein foul water 171 expelled in the separating plant 98, 104, 114 is used again at least as partial flow for mixing with the solids 116 so as to adjust an ideal dry matter content in the wet fermentation 164. For instance, the dry matter content may be between 5 and 15%. The excess of foul water 171 is added as circulating water to the waste water 170 of the wet fermentation 164 and can thus be supplied as diluting water 4 to the material solubilizer 1, for instance.

In accordance with the invention, the final concentration of the solids-bearing waste water 146 from the fermenter 138 in the separating plant 98, 104, 114 results in the fact that the solids content in the wet fermentation 164 can be optimally adjusted by the at least partial return of the solids-free foul water 171 to the expelled solids 116 and the wet fermentation reactor 192 can be dimensioned considerably smaller as well as the excessive solids-free foul water 171 can be injected in the circuit of the diluting water 4.

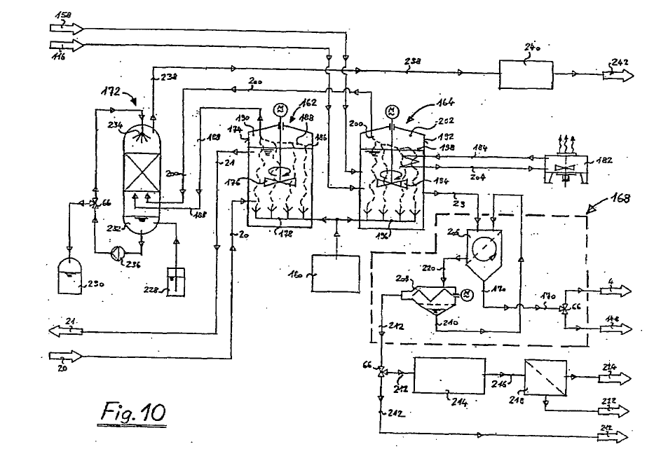

FIG. 10 shows a process diagram comprising the hydrolysis 162, the wet fermentation 164, the separating plant 168 as well as the acid pneumatic washer 172.

The decomposed suspension 20 is aerobically acidified by hydrolysis 162 and organic material is decomposed in such manner that it is likewise provided for fermentation in the fermenter 138. The adhesive grain and the pollutants are separated from the material which is not anaerobically decomposable.

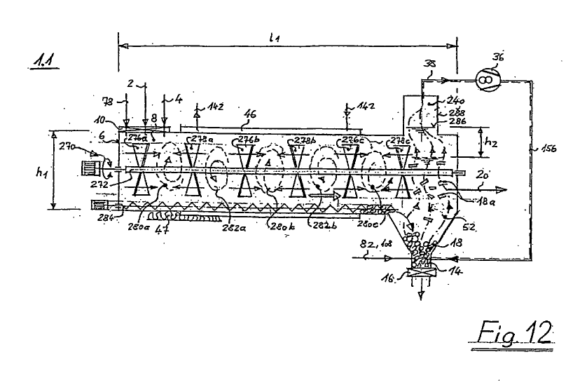

The hydrolysis 162 substantially comprises a reactor 174 in which a mechanical agitator 176 is arranged for mixing the material mixture (cf. FIG. 12). Near the bottom of the reactor 174a blowing means 178 for blowing in oxygen is provided which is fed through an oxygen supply 180. Above a material mixture level 186a waste gas chamber 188 is formed in which the waste gases 190 formed during hydrolysis 162 are collected.

The decomposed suspension 20 of the material solubilizer 1 is supplied to the reactor 174 near the bottom above the blowing means 178. The material mixture is mixed by the introduction of oxygen and by operating the agitator 176 and is discharged in the vicinity of the material mixture level 186 as treated suspension 21 after a treatment period of 1 to 4 days.

In the wet fermentation 164 the organic material which is not anaerobically decomposable is respired and the nitrogen is expelled as ammonia. In the wet fermentation 164 the circulating water 132, 133, 4 is NOₓ reduced by exposure to gas and thus a concentration of ammonium is prevented which disturbs the biology in the fermenter 138 and inhibits the gas production and decomposing performance.

The wet fermentation 164 substantially includes a reactor 192 in which an agitator 194 for mixing the material mixture 23 is arranged (cf. FIG. 12). Near the bottom of the reactor 192a blowing means 196 for blowing in oxygen is provided which is fed via the same oxygen supply 180 as that of the hydrolysis 162. Above a material mixture level 198a waste gas chamber 200 is formed for collecting the resulting waste gases 202.

In order to prevent the material mixture from overheating during wet fermentation 164a refrigerating unit 182 is provided. The refrigerating unit 182 is connected to an advance 184 and a return 204 immersing in the material mixture. For cooling the material mixture coolant is conveyed through the advance 184 and the return 204, whereby excessive heat in the material mixture can be discharged.

The solids 116 are filled into the reactor 192 in the vicinity of the agitator 194. In addition, the mixing water 158 strongly loaded with ammonia is introduced to the material mixture 192 above the solids 116. The material mixture is mixed by the agitator 194 and the introduced oxygen and, after a holding time of 3 to 10 days, is removed from the reactor 192 as treated and oxidized material mixture 23 and supplied to the separating plant 168.

The separating plant 168 comprises a filter means 206 and a dehydrating press 208. The treated and oxidized material mixture 23 is supplied to the filter means 206. The resulting almost solids-free waste water 170 is supplied to the diluting water 4 and/or the waste water treatment plant 148. Resulting solids and fibers 220 are further treated in the dehydrating press 208, for instance a classifying press. The filtrate 210 formed in the dehydrating press 208 is returned to the filter means 206. The dehydrated raw compost 212 formed can be subjected to subsequent fermentation and/or drying 214 via a change-over and/or dosing means 66.

In the subsequent fermentation 214 the dehydrated raw compost 212 is recovered into a separable dry product 216 having a dry matter content of 75% to 85%. The subsequent fermentation 214 is followed by a separating means 218 in which the inert material 222 is disposed of and the residual material 224 is supplied to material-sensitive recycling.

The waste gases 188, 200 collected in the waste gas chambers 190, 202 of the hydrolysis reactor 174 and the wet fermentation reactor 192 are supplied to a mixing container 226 of the acid pneumatic washer 172 and there are freed from ammonia. By charging hydrochloric acid or sulphuric acid 228, ammonium chloride or sulphate 230 can be obtained as commercial product. In the bottom area of the mixing container 226a water-acid mixture 232 accumulates which is removed from the mixing container 226 through a spraying means 234 having a circulating pump 236 and is sprayed in again at the top so that it can react with the waste gases 188, 200 all over the surface. Depending on the degree of treatment of the water-acid mixture 232, a part is removed during circulation via a change-over and/or dosing means 66 as finished commercial product ammonium chloride or sulphate 230. The NOₓ reduced waste air 238 resulting from this process can be freed from odorous substances in a connected cleaning step 240 and discharged to the atmosphere as purified process air 242.

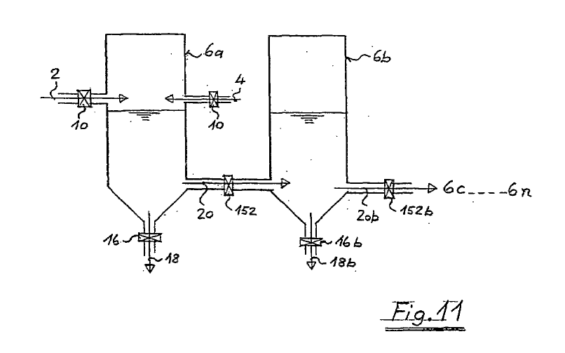

FIG. 11 shows a variant of a material solubilizer by which a so-to-speak continuous operation can be performed. In this embodiment two or more material solubilizing tanks 6 are connected in series, wherein each of them is designed to include a gas flow pump not shown in FIG. 10.

The mechanically treated input material 2 is supplied to the first material solubilizing tank 6a through the inlet lock 10 and is adjusted to the predetermined dry matter content by adding diluting water 4. The resulting impurities/high-gravity solids 18 are extracted through the outlet lock 16 disposed at the bottom and the decomposed suspension 20 resulting in the solubilizing tank 6a and intensely mixed by the pneumatic agitator is introduced to the further material solubilizing tank 6b by actuating a slide 152, wherein it is conveyed preferably without a pump by the action of gravity. In the latter tank it is further decomposed by means of the pneumatic agitator, wherein the resulting suspension 20b is then supplied via a slide 152 to one or more further solubilizing tanks (not shown) or to the treatment described by way of FIG. 9 by means of the fibrous material separator 98, the sand washer 122 and the fermenter 138. The impurities/high-gravity solids 18b formed in the material solubilizing tank 6b are again extracted at the bottom. The dry matter content TS is adjusted in the solubilizing tank 6b either in response to the dry matter content in the solubilizer 6a or solvent can be directly supplied to the solubilizing tank 6b so that the dry matter content can be adjusted individually in each material solubilizing tank 6a, 6b, . . . .

In FIG. 12 a basic structure of an alternative solubilizer 1.1 is shown in which organic material of the supplied input material 2 and/or of the screen underflow 78 of the screening plant 62 are dissolved in the diluting water 4. Preferably the solubilizer 1.1 according to FIG. 12 is used for the treatment of coarse residual waste and the solubilizer 1 according to FIG. 1 is used for the treatment of biological waste in mono batches. The minimum particle size of the waste mixture supplied (after mechanical treatment) is preferably 80 mm. The mixture 8 is diluted in the solubilizer 1.1 to a dry matter content of approx. 1 to 15%. The solubilizer 1.1 has a material solubilizing tank 6 of a rectangular shape which is in longitudinal section substantially “horizontal” and has a length L1 and a height h1. Preferably the height-to-length ratio h1:L1≧1:4 is satisfied.

The waste 278 and the diluting water 4 are supplied to the material solubilizing tank 6 via an inlet lock 10 in an end portion on the left according to representation. In an end portion of the solubilizing tank 6 on the right according to the representation in FIG. 12 a tapered bottom 12 is designed which opens into a discharge opening 14 having an outlet lock 16 through which the impurities/high-gravity solids 18 settling at the bottom 12 can be extracted. Above the tapered bottom 12a further outlet lock 16 is formed through which the suspension 20 decomposed in the material solubilizer 1 and loaded with organic material is extracted, treated according to the afore-described FIG. 9 and then supplied again as diluting water 4 via the inlet lock 10.

In the interior of the material solubilizing tank 6 an agitator 270 comprising a motor-driven rotor 272 is disposed which extends substantially over the entire length L1 of the material solubilizing tank 6 and on which a plurality of rotor blades 276a, b, c, 278a, b, c are arranged. Preferably an even number of rotor blades 276, 278 is chosen. The shown embodiment illustrates six rotor blades 276, 278, for instance, but also other numbers are imaginable.

The rotor blades 276, 278 have blade pitch angles offset by approx. 180° so that the rotor blades 276a, 278a and 276b, 278b and 276c, 278c each have opposite conveying directions. Thus, the mixture 8 is merged between the rotor blades 276a, 278a and 276b, 278b and 276c, 278c, whereby abrasion-promoting swirls 280a, 280b, 280c are formed and the organic material goes into solution. At the same time, between the rotor blades 278a, 276b and 278b, 276c a counter-swirl 282a, 282b is formed by which the mixture is guided apart and thus likewise the abrasion is promoted and the reaction of the organic material into solution is assisted. The impurities/high-gravity solids 18 settle downwards in the mixture and are conveyed, e.g., via a screw conveyor 284, to the tapered bottom 12 and thus to the outlet lock 16.

In order to release the organic material partly adhering to the impurities/high-gravity solids 18 completely from the latter a gas injecting means is provided by which preferably compressed air is blown into the discharge opening 14 by means of an injecting line 156 and an pneumatic compressor 36 in pulses, i.e. discontinuously, or continuously, whereby the impurities/high-gravity solids 18 rise to a particular distance h2 from the mixture level 286. The distance h2 can be variably chosen by the amount and the intensity of gas injection. The entire interior of the material solubilizing tank 6 is preferably filled with the mixture 8, wherein at a ceiling section opposed to the bottom 12a chimney 288 is disposed in which the mixture 8 rises. Above the mixture level 286a gas discharge chamber 240 which is connected to the pneumatic compressor 36 via a suction line 38 is formed in the chimney 288 so that the compressed air 52 of the gas injecting means can be circulated.

Moreover, in the area of the discharge opening 14 the process water 82 of the waste water treatment plant 148 as well as the circulating water 108 branched off the treatment circuit for the diluting water 4 can be introduced to the material solubilizing tank 6 so that the impurities/high-gravity solids 18 can leave the material solubilizing tank 6 as purified or clarified solids.

For adjusting an optimum processing temperature in the material solubilizing tank 6 the latter can be enclosed at least in portions by a double shell 46 through which a heating medium 142 is guided. In addition, insulation 47 enclosing the material solubilizing tank 6 and the double shell 46 can be provided.

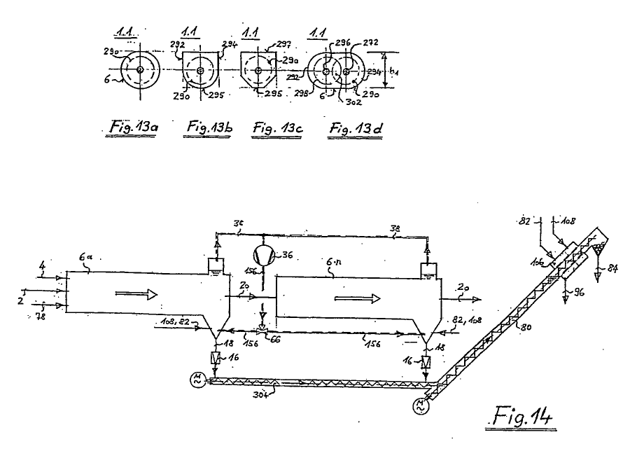

The FIGS. 13a-d illustrate exemplary cross-sections of the material solubilizer 1.1 from FIG. 12. The circle 290 shown in broken lines denotes the circular path described by the rotor blades 276, 278 with their tips.

It is imaginable according to FIG. 13a to design the material solubilizing tank 6 to have a circular cross-section or, according to FIG. 13b, to have two parallel longitudinal walls 292, 294 which are interconnected by a semi-circular bottom wall 295. It is equally possible to design the material solubilizing tank 6 according to FIG. 13c as polygon, especially as hexagon, wherein a bottom wall 295 has a shorter transverse extension than an opposite ceiling wall 297. In FIG. 13d a material solubilizing tank 6 having a rectangular cross-section with arc-shaped longitudinal walls 292, 294 is realized, wherein two rotors 274, 296 extending in parallel, whose rotor blade tips describe respective circular paths 290, 298 which together form an overlapping area 302, are arranged in the interior of the material solubilizing tank 6.

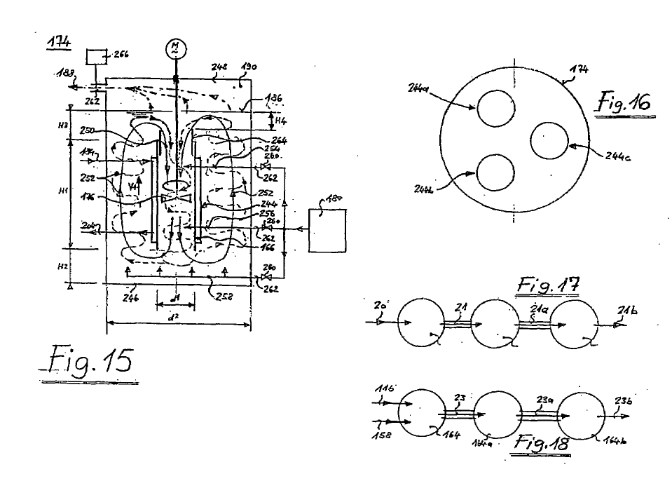

According to FIG. 14 plural material solubilizers 1.1 can be connected in series, the connected material solubilizing tank 6 being charged with the suspension 22 formed in the upstream material solubilizing tank 6. The gas injection is preferably effected by a common pneumatic compressor 36. The extracted impurities/high-gravity solids 18 are preferably supplied to the washing means 80 and, thus, to the further processing steps according to FIG. 9 by a common conveyor 304, for instance a screw conveyor. As an alternative to FIG. 9, the circulating water 108 can be introduced to the purification zone 106 of the washing means 80.

FIG. 15 shows a preferred embodiment of a hydrolysis reactor 174 for waste mixtures having a maximum particle size of approx. 80 mm. The wet fermentation reactor 192 is substantially designed for such particle sizes like the hydrolysis reactor 174 so that the following explanations are applicable to this reactor 192 and to the wet fermentation 164, too.

The reactor 174 for the hydrolysis 162 includes in the interior an agitator 176 having an adjustable conveying performance, preferably a blade agitator. The agitator 176 is enclosed by a double-walled draft tube 244 which is spaced apart on the front from the reactor bottom 146 and the reactor head 248 and preferably completely immerses in the material mixture. The agitator 176 is controlled in such manner that a circulating flow 250 is resulting, the material mixture in FIG. 15 being conveyed from the top to the bottom through the draft tube 244 and outside the draft tube 244a rising loop-shaped flow 252 being formed.

The draft tube 244 includes between its inner wall and its outer wall an annular chamber 166 connected to an upper advance 184 and a lower return 204 of a not represented refrigerating unit. When controlling the refrigerating unit, coolant flows through the annular chamber 166, whereby an overheating of the material mixture can be prevented.

An oxygen supply 180 is provided which optionally can blow oxygen into the material mixture via arms 254, 256, 258 near the bottom or in the area above and below the agitator 176. The arms 254, 256, 258 can have a plurality of gas injecting nozzles and are individually controlled to open and close by valves 262. The oxygen required for hydrolysis 162 can be provided both as liquid technical oxygen, i.e. <95% O₂ and can be treated in an air decomposition plant as enriched oxygen, i.e. <95% O₂. In the case of low-load material mixtures it is likewise possible to blow ambient air from the atmosphere into the reactor 174.

In the head area of the reactor 174a waste gas chamber 190 is formed for collecting the waste gases 188 formed during hydrolysis 162. The waste gas chamber 190 is delimited by the material mixture level 186. The waste gases 188 can flow off to the acid pneumatic washer 172 via a pipe 262 in the reactor head 248.

The oxygen bubbles moving upwards with the loop-shaped flow 252 can be sucked again by the agitator 176 through an axial extension 264 of the draft tube 244 adjustable in length and arranged at the top so that an almost 100% utilization of the provided oxygen is realized.

The oxygen utilization can be regulated via an O₂ probe 266 in the pipe 262 by defining the blown oxygen and the adjustment of the extension 264. It is also possible, however, to optimize the oxygen utilization via an axial displacement of the entire draft tube 244 and/or via a change of the material mixture level 186.

Hereinafter preferred conditions for optimized oxygen utilization are mentioned by way of example:

The height H1 of the draft tube corresponds to 8 to 10 times the diameter d1 of the draft tube.

The bottom distance H2 from the reactor bottom 246 to the draft tube 244 corresponds to 1 to 2 times the draft tube diameter d1.

The distance between the material mixture level 186 and the draft tube 244 corresponds to 2 to 3 times the draft tube diameter d1.

The variable height adjustment H4 between the material mixture level 186 and the draft tube 244 amounts to 0.5 to 2 times the draft tube diameter d1.

The upstream velocity v1 of the circulating flow 250 ranges between 0.1 m/s and 0.8 m/s.

The draft tube diameter d1 is between 0.5 m and 1.5 m depending on the material mixture composition and the dry matter content.

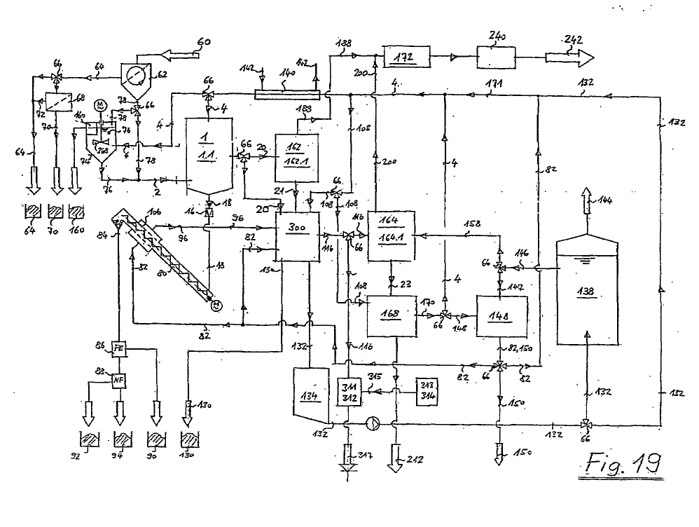

According to FIG. 16 also plural afore-mentioned draft tubes 244 can be provided in the reactor 174. So, for instance, three draft tubes 244a, 244b, 244c can be arranged in a triangle.

In accordance with FIGS. 17 and 18, it is imaginable for optimizing the hydrolysis 162 and the wet fermentation 164, respectively, to connect plural reactors 174 and 192 in series. The treated substance 21, 21a, 21b and/or the oxidized material mixture 23, 23a, 23b is subjected to a repeated hydrolysis 162a, 162b and/or wet fermentation 164a, 164b. The reactors 174 and 192 can also be operated in parallel, however.

In FIG. 19 a second process diagram for waste treatment of waste having organic components is schematically shown. The reference numerals are chosen in accordance with the first process diagram according to FIG. 9 so that, to avoid repetition, a detailed consideration of the common means and material flows is dispensed with.

At the beginning of the waste treatment the waste 60 to be treated is first supplied to a screening plant 62 which is a rotating screen, for instance. The waste 60 preferably has a dry matter content of 45 to 60%. The resulting screen overflow 64 can be disposed of either directly or can be supplied at least as partial flow to an air sizing plant 68 for separation of the screen overflow 64 into impurities/high-gravity solids 70 and soiled light solids 72 which then can be removed.

The screen underflow 78 rich in organic material can be supplied at least as partial flow to a mixing plant 74 in which it is diluted with a partial flow of NOₓ reduced diluting water 4 and recovered into a suspension 76 having a solids content of 5 to 15% by means of a mixer 268. Moreover, via a mechanical device of the mixing plant 74 impurities 160 such as ribbons, ropes and cables, are separated from the suspension 76 and ejected. The suspension 76 thus treated and freed from the coarse impurities 160 is supplied to the inlet lock 10 of the material solubilizer 1 or 1.1.

The impurities/high-gravity solids 18 contained in the material solubilizer 1, 1.1 are extracted to the material solubilizing tank 6 through the outlet lock 16 and are supplied to a washing means 80 in which the impurities/high-gravity solids 18 are purified from adhering organic material in a purification zone 106 by means of supplied process water 82. The impurities/high-gravity solids 18 purified in this way can then be supplied to a ferrous metal separator 86 as well as a non-ferrous metal separator 88 so that the material flow of the impurities/high-gravity solids 84 is divided into an iron-containing component 90 and a non-ferrous metal component 92 and other substances 94.

The decomposed suspension 20 extracted from the material solubilizer 1, 1.1 via the outlet lock 16 is subjected to a hydrolysis 162 or 162.1. Preferably in the hydrolysis 162, 162.1 a dry matter content of 5 to 15% is adjusted. The waste gases 188 loaded with nitrogen of the hydrolysis 162, 162.1 are supplied to an acid pneumatic washer 172 for NOₓ reduction and are subsequently discharged to the atmosphere as purified process air 240 after passing a purifying step 240 for freeing the NOₓ reduced waste gases from odorous substances.

The suspension 21 treated in the hydrolysis 162, 162.1 is supplied to a material separating plant 300 for separating the liquid 132 highly loaded with organic material from the solids 116 of the suspension 21 which are substantially free of organic material. So-to-speak as a side-product, purified fine sand 130 is resulting from this material separation which can be removed from the process.

The liquid 132 is stored in an intermediate reservoir 134 and is supplied, according to requirements, to a fermenter 138 for recovering biogas and/or to a heat exchanger 140 as circulating water by heating it to processing temperature by a heating medium 142 and then using it as diluting water 4 for the material solubilizer 1, 1.1.

The solids 116 preferably have a dry matter content of 5% and are subjected to a wet fermentation 164 or 164.1—also referred to as wet oxidation—. The waste gases 200 resulting from the wet oxidation 164, 164.1 and from the accompanying NOₓ reduction are strongly loaded with nitrogen and are fed to the acid pneumatic washer 172 for NOₓ reduction.

The material mixture 23 oxidized in the wet oxidation 164, 164.1 is supplied to a separating plant 168 from which, on the one hand, raw compost 212 is separated and, on the other hand, solids-free waste water 170 is supplied as diluting water 4 to the material solubilizer 1, 1.1 and/or purified in a waste water treatment plant 148 for discharging it as waste water 150 into the sewerage system. A partial flow of the purified waste water 150 is guided as process water 82 into the purification zone 106 of the washing means 80 as well as to the material separation plant 300. Likewise a partial flow of the purified waste water 150 is mixed as process water 82 with the partial flow of the circulating water 132 after the fermenter 138.

In the fermenter biogas 144 is obtained from the organically highly loaded circulating water 132 by the action of methane bacteria. Therefrom unloaded waste water 146 is resulting which can be supplied as unloaded foul water 159 to the wet oxidation 164, 164.1. The material flow of the waste water 146 not required for the wet oxidation 164, 164.1 can be supplied to the waste water treatment plant 148 as excessive water 174.

Furthermore it is shown in FIG. 19 that the dehydrated solids 116 can be supplied at least as partial flow after passing a drying 311 to a compacting plant 312 for producing a fuel for thermal/material-sensitive recycling in a gasification or combustion plant 317, wherein a binder 315 prepared in a liquefying means 313 and/or a preparing or dosing means 314 is supplied to the compacting plant 312 for use as adhesive.

Hereinafter a detailed description of the hydrolysis 162.1, the wet oxidation 164.1, the material separating plant 300, the separating plant 168 as well as the compacting is given.

During hydrolysis 162.1, as already during the hydrolysis with the reactor according to FIG. 15, the decomposed suspension 20 is roughly cleaned and organic material is decomposed such that it is available for fermentation in the fermenter 138. Furthermore, the not anaerobically degradable are separated from adhesive grains and pollutants.

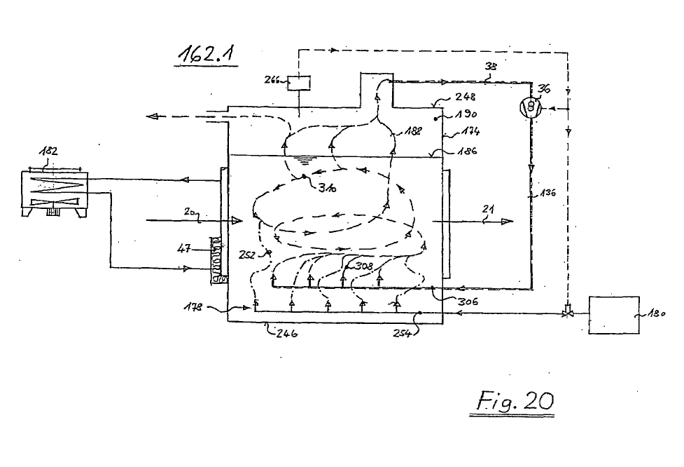

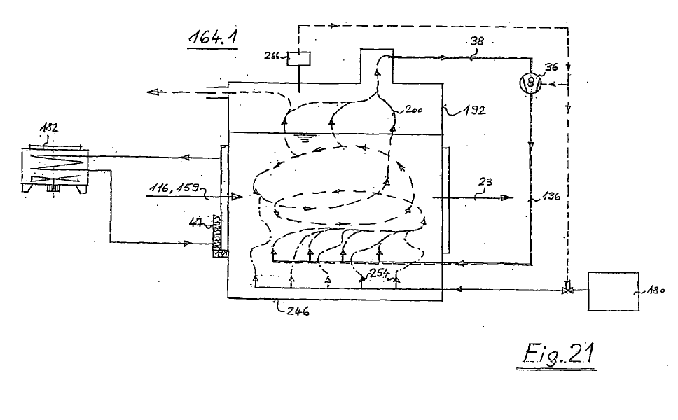

According to FIG. 20, for material mixtures having a minimum particle size of approx. 80 mm the hydrolysis is substantially performed in a reactor 174 having near the bottom 246a blowing means 178 for blowing in oxygen, whereby a helical flow 252 ascending in the material mixture is formed by which the material mixture is mixed. Accordingly, no mechanical agitator is necessary. The blowing can be carried out in pulses or continuously.

The charging of the reactor 174 with the suspension 20 from the material solubilizer 1, 1.1 as well as the discharge of the hydrolyzed suspension 21 take place in a central reactor section.

The blowing means 178 comprises at least one lance or one arm 254 having a plurality of nozzles which is connected to an oxygen supply 180 for blowing the oxygen into the material mixture. Preferably pure oxygen is blown in through the nozzles.

The blown oxygen and the waste gases 188 resulting from the hydrolysis 162.1 accumulate above a material mixture level 186 in a waste gas chamber 190. Since during hydrolysis 162a part of the oxygen is respired, i.e. rendered inert, by CO₂, an O₂ measuring probe 266 is provided in the reactor top 248 for optimum control of the oxygen supply 180.

For improving the thorough mixing of the material mixture in the hydrolysis reactor 174 at least a partial flow of the waste gas 188 can be injected via a suction line 38, an pneumatic compressor 36, an injecting line 136 as well as an arm 306 provided with a plurality of nozzles and arranged, according to the view in FIG. 20, above the lance 254 of the blowing means 178 into the material mixture in pulses or continuously. The injected waste gases 188 likewise form an ascending helical flow 308 which is superimposed to the flow 252 of the injected oxygen into a total flow 310.

The waste gases 188 not injected into the material mixture are supplied to the acid pneumatic washer 172 for NOₓ reduction, as already described under FIG. 19.

The dry matter content of the material mixture preferably amounts to 5 to 15% and the temperature of the material mixture in the reactor 174 is 70° C. Said temperature is sufficient to dissolve fat and/or fat compounds. In order to constantly maintain 70° C. the insulation 47 is provided through which coolant of a refrigerating unit 182 flows.

In the wet fermentation or wet oxidation 164.1, as in the wet fermentation 164 including the wet fermentation reactor according to FIG. 15, the not anaerobically degradable organic material is respired and the nitrogen is expelled as ammonia. During oxidation 164.1 the circulating water 132, 133, 4 is NOₓ reduced by exposure to gas and thus a concentration of ammonium is prevented which disturbs the biology in the fermenter 138 and inhibits the gas production and the decomposing performance.

The wet oxidation 164.1 is carried out for material mixtures having a minimum particle size of approx. 80 mm according to FIG. 21 substantially in a reactor 192 corresponding to the reactor 174 of the hydrolysis 162.1. Said reactor 192, too, shows a blowing means 178 which is close to the bottom and can be operated in pulses and discontinuously for blowing in oxygen and for mixing the material mixture in the reactor 192. An above-described O₂ measuring probe 266 is provided for the control of the oxygen supply 180.

Likewise the waste gases 200 formed during wet oxidation 164.1 can be injected again in pulses or discontinuously by return into the material mixture at least as partial flow. The non-returned waste gases 200 are supplied to the acid pneumatic mixer 172 for NOₓ reduction according to FIG. 19.

Further, insulation 74 by a refrigerating unit 182 is provided for adjusting a constant temperature of the material mixture.

Moreover, the supply of the solids 116 dehydrated in the material separation 200 as well as the foul water 159 of the fermenter 138 and the discharge of the oxidized material mixture 23 as the material flows 20, 21 during hydrolysis 162.1 are carried out in a central reactor section. Preferably a dry matter content of 5 to 15% is adjusted in the reactor 192. The supplied foul water 159 primarily serves as diluting water.

The substantial difference between the hydrolysis reactor 174 and the wet oxidation reactor 192 consists in the fact that during wet oxidation 164.1 more oxygen is injected into the material mixture to react the substances which have not yet gone into solution into the latter as well as to subject the material mixture to NOₓ reduction. This has the advantage that a subsequent fermentation 214, as in the process diagram according to FIG. 9 and FIG. 10, can be dispensed with, whereby considerable reductions of costs, inter alia, are possible.

Apart from respiring the non anaerobic degradable organic material and from expelling the nitrogen as ammonia, during wet oxidation 164, 164.1 likewise the material mixture can be hygienized in the reactor 192 depending on the type of control. In this context, not only the solids 116 provided in the material mixture but also the waste waters 146 of the fermenter 138 clogged with or without solids can be hygienized. Waste waters of composting plants can equally be hygienized with the aid of wet oxidation 164, 164.1.

Preferably the hygienization during wet oxidation 164, 164.1 is carried out at the beginning of the wet oxidation 164, 164.1, because at the prevailing high temperatures also an improvement of the microbial availability of the organic substances is brought about. It is also possible, however, to carry out the hygienization at the end of the wet oxidation 164, 164.1.

The hygienizing times are dependent on the prevailing temperatures so that, depending on the temperature, different hygienizing times have to be observed. For instance, a hygienizing performance required by the German biological waste regulation can be obtained at 70° C. over a period of one hour. At lower temperatures the holding time must be appropriately extended.

The hygienization is relevant especially to all biomass raw materials which are to be supplied to agricultural utilization. These include especially biological and green waste, waste from agriculture and energy plants, kitchen and canteen waste, sludge as well as specific process and waste waters. All over Europe also biomass products from the total refuse can be added hereto.

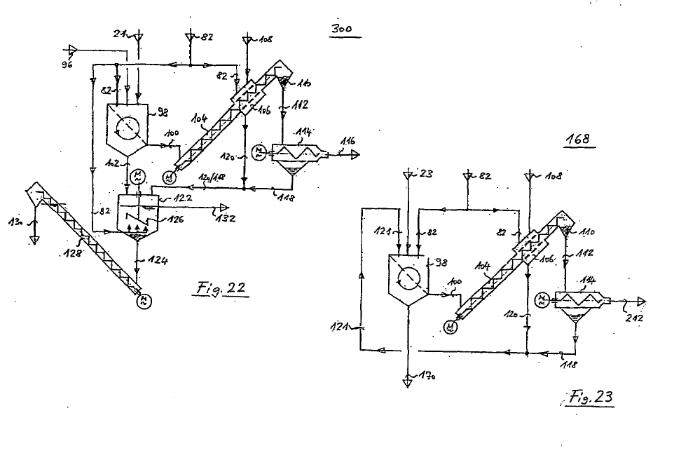

FIG. 22 shows a schematic structure of the material separation plant 300. The suspension treated in hydrolysis 162. 162.1 is supplied together with the soiled process water 96 from the washing means 80 to a fibrous material separator 98 which, by way of example, is in the form of a rotating screen. In addition, the process water 82 obtained in the waste water treatment plant 148 can be supplied to the fibrous material separator 98 as dilution. In the fibrous material separator 98 fibers and floating substances 100 are separated from the water 102 containing organic material.

The fibrous and floating substances 100 are purified in a solids screening and washing plant 104 by adding a partial flow of the process water 82 in a purification zone 106. This purifying operation can be assisted by the fact that additionally circulating water 108 branched off the circuit of the diluting water 4 upstream of the heat exchanger 140 is guided through the purification zone 106. In the purification zone 106 the organic components of the fibrous and floating substances 100 are dissolved therefrom. If a very intense purification is necessary, the process water 82 is additionally fed to the purification zone 106. In the case of less intense purifications, the share of circulating water 108 can be increased.

The purified solids and fibers 112 extracted through a solids outlet 110 of the washing plant 104 are dehydrated in a dehydrating press 114 and the dehydrated solids 116 are subjected to the wet oxidation 164, 164.1.

The water 118 resulting from the dehydrating press 114 and charged with organic material is supplied to a sand washer 122 together with the rinsing water 120 flowing off the purification zone 106 and charged with organic material. The water 102 containing organic material can also be supplied to the sand washer 122. In the sand washer 122 the fine sand component 124 is separated by the action of an agitator 126 and the organic components adhering to the fine sand component 124 are dissolved by the addition of the process water 82. The fine sand 124 pre-cleaned in this way is then fed to a fine sand washing means 128 the basic structure of which corresponds to the washing means 80 and 104 according to FIG. 19. The cleaned fine sand 130 then can be supplied to a material-sensitive utilization in civil engineering and road construction.

The liquid 132 resulting during sand washing and highly loaded with organic material is intermediately stored, as already described in FIG. 19, in the intermediate reservoir 134 and is fed to a fermenter 138 and/or used as circulating water 132.

According to FIG. 23, in the separating plant 168 the oxidized material mixture 23 of the wet oxidation 164, 164.1 is supplied to a fibrous material separator 98 together with the process water 82 and mixing water 121 from a solids screening and washing plant 104 and a dehydrating press 114 for obtaining the solids-free waste water 170 which is supplied to the waste water treatment plant 148 as described in FIG. 19 and/or is used as diluting water 4 for the material solubilizer 1, 1.1.

The fibrous material separator 98 is in the form of a rotating screen by way of example, wherein the separated fibrous and floating substances 100 are supplied to the solids screening and washing plant 104 in the purification zone 106 of which the adhering organic material is separated by means of the process water 82 and/or the branched off circulating water 108. The solids 112 dehydrated and cleaned after the purification zone 106 are extracted through a solids outlet 110 and are compressed in the dehydrating press 114 to form the raw compost 212 already mentioned in FIG. 19.

The water 118 highly loaded with organic material and pressed out in the dehydrating press 114 is supplied to the fibrous material separator 98 together with the rinsing water 120 of the solids screening and washing plant 104 as mixing water 121.

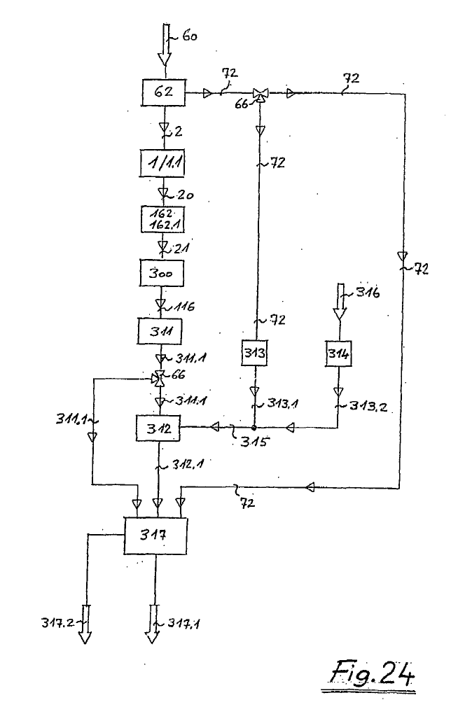

In accordance with FIG. 24, during compacting for the production of fuels for the gasification or combustion plant 317 from FIG. 19 the dehydrated solids 116 are subjected to drying 311. After drying 311, a resulting dry matter mixture 311.1 having a water content of preferably 15% to 25% is supplied to a compacting plant 312, especially a briquetting or pelleting means having an integrated mixer or extruder or a bar press. The compacting is preferably carried out under low pressure, wherein a binder is mixed to the dry matter mixture 311.1 as adhesive so as to keep the molded parts 312.1 produced under low pressure, such as e.g. briquettes or pellets, together until glowing away 317. The compacting under low pressure and with addition of the binder 315 has the advantage that the energy spent on producing the molded parts 317 is reduced and the wear of the component parts of the compacting plant 312 such as e.g. the mixer is reduced. So the compacting 312 according to the invention with the binder 315 requires approx. 20 kW electric current and incurs wear costs of about 1 C=/Mg to 6 C=/Mg, whereas in a conventional compacting for producing 1 Mg of molded parts from waste 100 kW electric current are needed and wear costs of about 15C= are incurred, whereby total costs/Mg of about 50C= are incurred.

The adhesive 315 is primarily obtained from the generated screen overflow 72 consisting at about 80% of plastic material and being formed in a liquefying apparatus 313 by extrusion or thermal/chemical action into a viscous injecting mass 313.1.

In case that no or too little plastic material 72 is available also supplied binder 316 such as e.g. lime milk or starch via the preparing and dosing means 314 can be added to the compacting plant 312 as organic or inorganic binder 314.1. In this case, of course the organic starch such as potato starch, for instance, is preferred, because the latter is burnt residue-free in contrast to the cheaper lime milk and electric and/or thermal energy 317.1 is released. The lime milk can be disposed of as slag or mineral substances 317.2.

Depending on the quality requirements to the fuel to be supplied to the gasification or combustion plant 317, the compacting plant 312 can be bypassed completely or partly and the material flows 72 and 311.1 can be directly fed to the thermal utilization 317.

A method is disclosed for the treatment of waste with organic components, whereby in standardized method steps, various material solubilizers, for dissolving the organic material in a solvent and various reactors for carrying out a hydrolysis and/or wet fermentation are used depending on the particle size and suitable solubilizers and reactors. A suitable waste treatment plant is also disclosed.

Although the best mode contemplated by the inventors of carrying out the present invention is disclosed above, practice of the present invention is not limited thereto. It will be manifest that various additions, modifications and rearrangements of the features of the present invention may be made without deviating from the spirit and scope of the underlying inventive concept.

1. A method for the treatment of waste with organic components, comprising the steps of:
mechanical treatment of the waste into a waste mixture,
dissolving organic components in a material solubilizer,
hydrolysis of the suspension extracted from the material solubilizer and loaded with organic material in a reactor and
fermentation of the hydrolyzed suspension in a fermenting step, wherein
the process water obtained during hydrolysis or fermentation is circulated as circulating water, and
selecting the material solubilizer and the reactor for the hydrolysis in response to the particle size of the mechanically treated waste mixture.

2. A method according to claim 1, wherein the material solubilizer and the reactor are changed in the case of a particle size of about 80 mm.

3. A method according to claim 1, wherein a wet fermentation or wet oxidation is connected downstream of the hydrolysis at least indirectly.

4. A method according to claim 1, wherein separating steps for separating impurities, high-gravity solids, fibrous substances etc. from biological suspension to be supplied to the fermenting step are provided.

5. A material solubilizer for use in the method according to claim 1 for dissolving organic components of waste in a solvent having particular maximum particle size of about 80 mm, comprising a material solubilizing tank in which a mixing means for mixing the waste and the solvent is arranged, wherein the suspension loaded with organic material is extracted via a suspension outlet, characterized in that the mixing means has at least one gas injecting nozzle through which a gas pressurizes the suspension such that organic components go into solution or are distributed in the solvent by the shear forces applied by the gas.

6. A material solubilizer according to claim 5, wherein the gas injecting nozzle is part of a gas flow pump by which the suspension can be recirculated periodically or continuously inside the material solubilizing tank.

7. A material solubilizer according to claim 6, wherein the pulse distance is more than 3 seconds, preferably between about 5 and 10 seconds.

8. A material solubilizer according to claim 6, wherein the gas flow pump has an inner pipe at the lower inlet opening of which a nozzle plate including a plurality of gas injecting nozzles around or through which the suspension flows is arranged and the upper end portion of which has an outlet opening for the suspension conveyed in the inner pipe.

9. A material solubilizer according to claim 8, wherein at a distance from the outlet opening of the inner pipe a bounce plate is disposed.

10. A material solubilizer according to claim 9, wherein the bounce plate delimits a gas discharge chamber at least in sections.

11. A material solubilizer according to claim 6, wherein plural gas flow pumps are arranged in the material solubilizing tank.

12. A material solubilizer according to claim 8, wherein the inner pipe is double-walled and the gas injecting nozzles are disposed in the inner cylinder chamber or in the annular chamber and a heating medium flows through the respective other chamber.

13. A material solubilizer according to claim 5, wherein the gas is at least one of guided in the circuit and is sucked from the material solubilizing tank by a pump, and (B) pressurized and returned from a reservoir to the gas injecting nozzles.

14. A material solubilizer according to claim 8, wherein in the annular chamber delimited by the inner pipe and by the outer circumferential wall of the solubilizing tank deflector plates are arranged for guiding the flow.

15. A material solubilizer according to claim 5, wherein plural material solubilizing tanks are connected in series and the suspension flows from the first solubilizing tank to the connected solubilizing tanks.

16. A material solubilizer according to claim 5, further comprising an extracting opening for impurities/high-gravity solids.

17. A material solubilizer according to claim 5, wherein a connection for gas injection and mixing the settled impurities/high-gravity solids is provided in the discharge opening.

18. A material solubilizer according to claim 5, wherein the solvent is circulated.

19. A material solubilizer for use in the method according to claim 1 for dissolving organic components and waste in a solvent having a particular minimum particle size of about 80 mm, comprising a material solubilizing tank in which at least one agitator for mixing the waste and the solvent into a suspension is arranged, wherein the suspension loaded with organic material is extracted through an outlet lock, characterized in that the agitator includes a plurality of adjacent agitating elements which show respective opposed conveying directions.

20. A material solubilizer according to claim 19, wherein the agitating elements are rotor blades arranged on a common rotor and adjacent rotor blades have a blade pitch angle offset by about 180°.

21. A material solubilizer according to claim 20, wherein the rotor blades are evenly arranged on the rotor from an inlet opening to an outlet lock for impurities/high-gravity solids.

22. A material solubilizer according to claim 20, wherein an even number of rotor blades is chosen.

23. A material solubilizer according to claim 20, wherein two rotors are provided which form an overlapping area with their rotor blades.

24. A material solubilizer according to claim 19, wherein in the area of a discharge opening for the impurities/high-gravity solids a gas can be blown in.

25. A material solubilizer according to claim 24, wherein the gas is circulated and is sucked by a pump from the material solubilizing tank and returned to the same.

26. A material solubilizer according to claim 19 to, wherein plural material solubilizing tanks are connected in series and the suspension flows from the first material solubilizing tank into the connected material solubilizing tanks.

27. A material solubilizer according to claim 19, wherein the material solubilizing tank in the longitudinal section has a substantially rectangular shape the height-to-length ratio corresponds to the equation h1:L1≧1.4.

28-62. (canceled)
